(19) United States
(12) Patent Application Publication
(10) Pub. No.: US 2013/0327337 A1
(43) Pub. Date: Dec. 12, 2013

(54) Neonatal Endotracheal Tube with Monolithic Secure Guard

(71) Applicants: Srisatish Devapatla, Ithaca, NY (US); Madhavi D. Reddy, Ithaca, NY (US)

(72) Inventors: Srisatish Devapatla, Ithaca, NY (US); Madhavi D. Reddy, Ithaca, NY (US)

(21) Appl. No.: 13/492,254

(22) Filed: Jun. 8, 2012

(51) Int. Cl. A61M 16/04

(52) U.S. Cl. 128/207.14

(57) ABSTRACT

A secure guard for an endotracheal tube includes a base ring, and a short gripper, a long gripper, and an upper lip bar each extending from the base ring. The short gripper and the long gripper each includes a flexible neck extending from the base ring and a gripping portion extending from the flexible neck. Each gripping portion includes a first gripping projection and a second gripping projection forming an opening separated by a gap to permit insertion of the endotracheal tube laterally into the opening. The gripping portions preferably include projections to contact the endotracheal tube and prevent sliding of the tube with respect to the guard. The upper lip bar includes an upper lip bar body and an upper lip adhesive strip coupled to the upper lip bar body. In some embodiments, the secure guard is sized to fit a neonate.

### BACKGROUND OF THE INVENTION

1. Field of the Invention

The invention pertains to the field of neonatal medicine. More particularly, the invention pertains to an endotracheal tube and a guard for an endotracheal tube.

2. Description of Related Art

When a neonate needs endotracheal intubation for any medical indication, quick and safe intubation is extremely important. The endotracheal (ET) tube tip needs to be positioned in the trachea above the carina. It is extremely difficult for a physician without intubation experience to properly insert an ET tube the correct distance into a patient the first time without assistance. Placing the ET tube at the carina or into the bronchi can lead to complications such as contralateral lung collapse, pneumothorax, bradycardia, and potentially death.

In order to prevent the ET tube placement deep into the bronchi, the following conventional methods are adopted:

1. The ET tube is marked just above the tip of the tube to guide placement of the tube to an optimum depth. The vocal cords should be located at the level of this mark when the tube has been inserted to the optimum depth.

2. The sum of six and the weight of the neonate in kilograms (kgs) gives the length of the lip level, in centimeters, in order to place the end of the ET tube just above the carina.

In actual practice, many times the ET tube is inserted too far such that after intubation the neonates have the ET tube in the right main stem bronchi. This often leads to one or more complications such as contralateral lung collapse, pneumothorax, and potentially death without timely intervention. If the ET tube is found to be in an incorrect position by chest x-ray, it is typically adjusted. The ease with which an ET tube can be adjusted depends on how it is secured to the baby's skin. Accidental extubation can happen during tube readjustment.

Guards for endotracheal tubes are known in the art.

U.S. Pat. No. 5,060,647, entitled “Short Self-Adhesive Denture Guard” and issued Jul. 23, 1991 to Wiley et al., discloses a dental guard fittable over a patient's upper incisors composed of an arcuate plastic tray of about 1.5 inches in length and having in cross-section a U-shape. The dental guard protects the incisors from the levering forces exerted by the laryngoscope blade during intubation and provides the person intubating with an unobstructed view for guiding an endotracheal tube via the pharynx through the vocal cords and into the trachea.

U.S. Pat. No. 7,866,313, entitled “Oral Airways that Facilitate Tracheal Intubation” and issued Jan. 11, 2011 to Isenberg et al., discloses an oral airway including a first component and a second component adapted to be removably coupled together such that a first guiding surface and a second guiding surface collectively define and encompass an interior passage through the oral airway that is dimensioned to direct a fiber-optic scope or an endotracheal tube extending through the interior passage for tracheal intubation. The first and second components are configured to be decoupled and independently removed from a patient's mouth without disrupting an endotracheal tube that has been extended through the conduit for tracheal intubation. The first and second components may be maintained in coupled disposition by an interlocking mechanical structure or by magnetism.

U.S. Patent App. Pub. No. 2009/0255538, entitled “Monolithic Endotracheal Tube Holder”, by Thomson et al., published Oct. 15, 2009 and discloses a device for retaining a medically relevant tube into a proper registration for application of medical treatment to a patient, and more particularly to a flexible holder for positioning of an endotracheal tube. The holder has integrated circumferential guard projections for maintaining optimal flow characteristics of the restrained endotracheal tube. Circumferential guard projections extend both outwardly and inwardly from the endotracheal tube holder and are integral to and monolithically formed with the endotracheal tube holder. The endotracheal tube holder is adaptable to receiving tubes of varying diameters and includes a capture means for allowing insertion and removal of an endotracheal tube through a transverse access port in a side aspect of the holder. The monolithic nature of the endotracheal tube holder design is further enhanced through incorporation of access portals about the holder for allowing routine patient maintenance.

U.S. Patent App. Pub. No. 2011/0132380, entitled “Mouth Guard”, by Goldsby, published Jun. 9, 2011 and discloses a mouth guard with a top mouth piece for engaging the top teeth and a bottom mouth piece for engaging the bottom teeth. The mouth guard also has a central section with one or more ports for receiving an endotracheal tube, an oral gastric tube, or an oral suction tube. A top portion of the central section is attached to the top mouth piece and a bottom portion of the central section is attached to the bottom mouth piece, or the entire central section is attached to the bottom mouth piece. The top and bottom mouth pieces are interlocking via male engaging members and female recesses.

### SUMMARY OF THE INVENTION

The endotracheal tube includes a secure guard and a protection ring. The endotracheal tube inserts into a base ring on the secure guard. A short gripper, a long gripper, and an upper lip bar each extend from the base ring. The short gripper and the long gripper each includes a flexible neck extending from the base ring and a gripping portion extending from the flexible neck. Each gripping portion includes a first gripping projection and a second gripping projection forming an opening separated by a gap to permit encircling and firmly holding the endotracheal tube. The gripping portions preferably include projections to contact the endotracheal tube and prevent sliding of the tube with respect to the guard. The upper lip bar includes an upper lip bar body and an upper lip adhesive strip coupled to the upper lip bar body. In some embodiments, the secure guard is sized to fit a neonate. The protection ridge, a small circumferential plastic projection from the outer surface of the endotracheal tube at the 6 centimeter (cm) level, is positioned below the secure guard and prevents slipping of the secure guard into the neonate's oropharynx.

### BRIEF DESCRIPTION OF THE DRAWINGS

FIG. 1 shows a top view of a monolithic secure guard including a base ring, a short gripper, a long gripper, and a horizontal upper lip bar with a hydrocolloid gel strip protected by a cover strip in an embodiment of the present invention.

FIG. 2 shows a side view of the monolithic secure guard of FIG. 1.

FIG. 3a shows a front view of the upper lip bar of the monolithic secure guard of FIG. 1 with the cheek extension hydrocolloid gel strip protected by a cover strip.

FIG. 3b shows a back view of the upper lip bar of FIG. 3a.

FIG. 4 shows the monolithic secure guard of FIG. 1 with the short gripper encircling an ET tube having a protection ridge at the 6 centimeter (cm) level.

FIG. 5 shows the monolithic secure guard and ET tube of FIG. 4 with both the short gripper and the tall gripper encircling the ET tube.

FIG. 6 shows the monolithic secure guard and ET tube of FIG. 5 applied to a neonate using the adhesive hydrocolloid gel strips.

FIG. 7 shows a perspective view of a monolithic secure guard in an embodiment of the present invention.

### DETAILED DESCRIPTION OF THE INVENTION

The endotracheal tube with the monolithic secure guard preferably prevents endobronchial intubation and assists in quick and safe securing of an endotracheal (ET) tube. Although the endotracheal tube with the monolithic secure guard may be sized for use on any patient, the endotracheal tube with the monolithic secure guard is preferably sized for use on a neonate. A neonate, as used herein, may refer to any newborn human infant, including, but not limited to, full-term infants under one month of age and premature infants. The ET tube preferably has an elevated ridge built in the outer wall at a distance 6 centimeters (cm) from the tip. This elevated ridge prevents accidental slipping of the monolithic secure guard into the neonate's mouth. ET tubes are conventionally made of polyvinyl chloride (PVC) with a hockey stick shape for insertion. The monolithic secure guard has an upper lip bar. The upper lip bar secures the monolithic secure guard to the upper lip of the patient and is preferably made of soft plastic with an adhesive strip on the surface facing the neonate's upper lip and extending on to the top of the upper lip bar to form cheek extension strips. The adhesive strip is preferably made of a hydrocolloid gel. A cover strip, which may be a plastic strip of paper, protects the sticky surface of the hydrocolloid gel strip until ready for use. This cover strip with a tab is peeled off to stick the adhesive surface of the gel strip to the skin above the neonate's upper lip. A cheek extension strip is also used for safe securing of the ET tube. The cheek extension strip includes an adhesive strip that is preferably made of a hydrocolloid gel.

A base ring is attached to the upper lip bar. This base ring is used to hold the ET tube and forms the base for the two grippers. In some embodiments, the two grippers are a short gripper and a long gripper that are perpendicular grippers extending to grip on opposite sides of the ET tube. In some embodiments, the long gripper is located on the upper lip side and the short gripper is located on the lower lip side. In some embodiments, the long gripper is located on the right cheek side and the short gripper is located on the left cheek side. In some embodiments, each gripper has a narrow flexible base attached to the base ring and a wider top with a circular inner surface with small projections. In some embodiments, the projections are formed monolithically with the gripper bodies. In some embodiments, the projections are made of plastic. In other embodiments, the projections are made of metal. The projections grip the ET tube and prevent it from slipping with respect to the secure guard. In some embodiments, the cover strip on the upper lip bar, facing away from the baby has the ET tube intubation depth formula.

In some embodiments, the base ring, the horizontal upper lip bar except for the hydrocolloid gel strip and cover strip, the short gripper, the long gripper, and the projections are formed monolithically. In some embodiments, the base ring, the horizontal upper lip bar except for the hydrocolloid gel strip and cover strip, the short gripper, the long gripper, and the projections are formed monolithically of PVC.

Neonatal size varies significantly based on their gestational age and weight. A measurement of the upper lip distance from the left angle of the mouth to the right angle of the mouth is preferably collected on the baby weighing between 500 grams and 4500 grams prior to the selection and use of the endotracheal tube with the secure guard. In some embodiments, the endotracheal tube with the monolithic secure guard is designed in 4 sizes: 2.5 French (Fr) for a birth weight less than 1 kilogram (kg) and/or a gestational age<28 weeks, 3.0 Fr (a birth weight between 1 and 2 kgs and/or a gestational age 28 to 34 weeks), 3.5 Fr (a birth weight between 2 and 3 kgs and/or a gestational age 34 to 38 weeks), and 4.0 Fr (a birth weight>3 kgs and/or a gestational age>38 weeks). In some embodiments, the ET tube has the monolithic secure guard in place. In some embodiments, the base ring of the secure guard is at the following cm mark based on the size of the ET tube: 8 cm for the 2.5 Fr ET tube, 9 cm for the 3.0 Fr ET tube, 10 cm for the 3.5 Fr ET tube, and 11 cm for the 4.0 Fr ET tube.

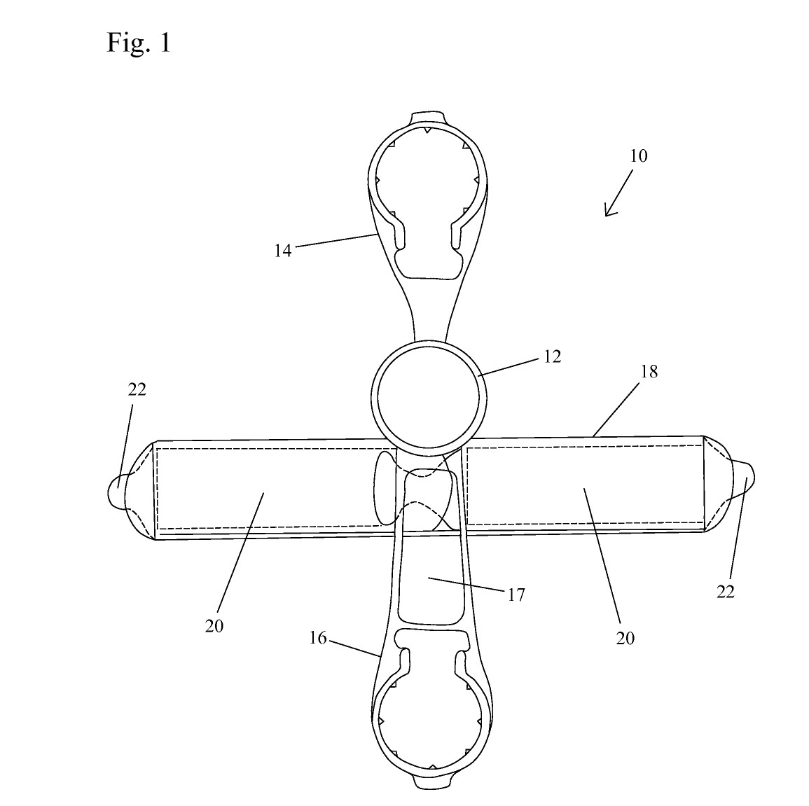

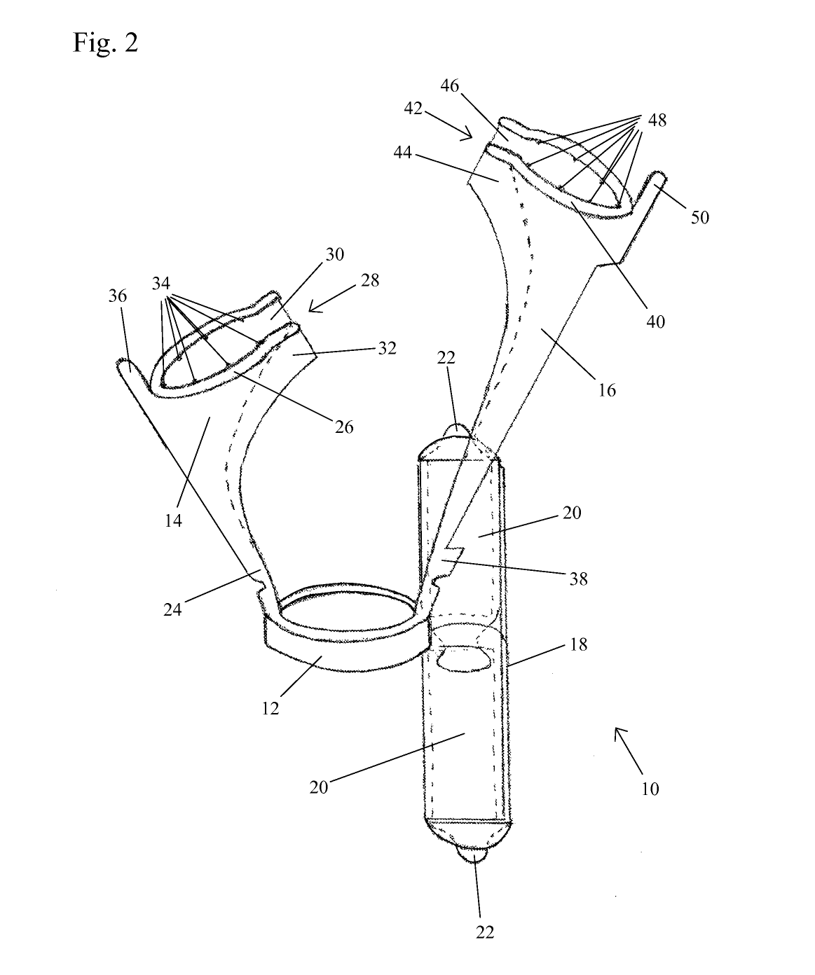

FIG. 1 and FIG. 2 show a top view and a side view, respectively, of a secure guard 10. The secure guard includes a base ring 12. A short gripper 14, a long gripper 16, and a horizontal upper lip bar 18 extend from the base ring 12. The horizontal upper lip bar 18 includes at least one hydrocolloid gel strip 20 protected by at least one cover strip 22. The secure guard 10 is monolithic in that all but the hydrocolloid gel strip 20 and the cover strip 22 are formed as a single piece of plastic.

The short gripper 14 includes a flexible neck 24 connecting a gripping portion 26 to the base ring 12. The gripping portion 26 has a gap 28 to receive an ET tube, a pair of flexible gripper tabs 30, 32 to slide laterally around the ET tube, and a plurality of projections 34 extending inwardly from the gripping portion 26 to grip the ET tube. A finger tab 36 extending from the back of the gripper portion 26 aids in removal of the gripper portion 26 from an ET tube by giving the user something to grab or push.

The long gripper 16 includes a flexible neck 38 connecting a gripping portion 40 to the base ring 12. The long gripper 16 flexible neck 38 is longer than the short gripper 14 flexible neck 38 such that the long gripper 16 gripping portion 40 grips the ET tube at a position farther from the position of the base ring 12 than where the short gripper 14 gripping portion 26 grips the ET tube. The gripping portion 40 has a gap 42 to receive an ET tube, a pair of flexible tabs 44, 46 to slide laterally around the ET tube, and a plurality of projections 48 extending inwardly from the gripping portion 40 to grip the ET tube. A finger tab 50 extending from the back of the gripper portion 40 aids in removal of the gripper portion 40 from an ET tube by giving the user something to grab or push.

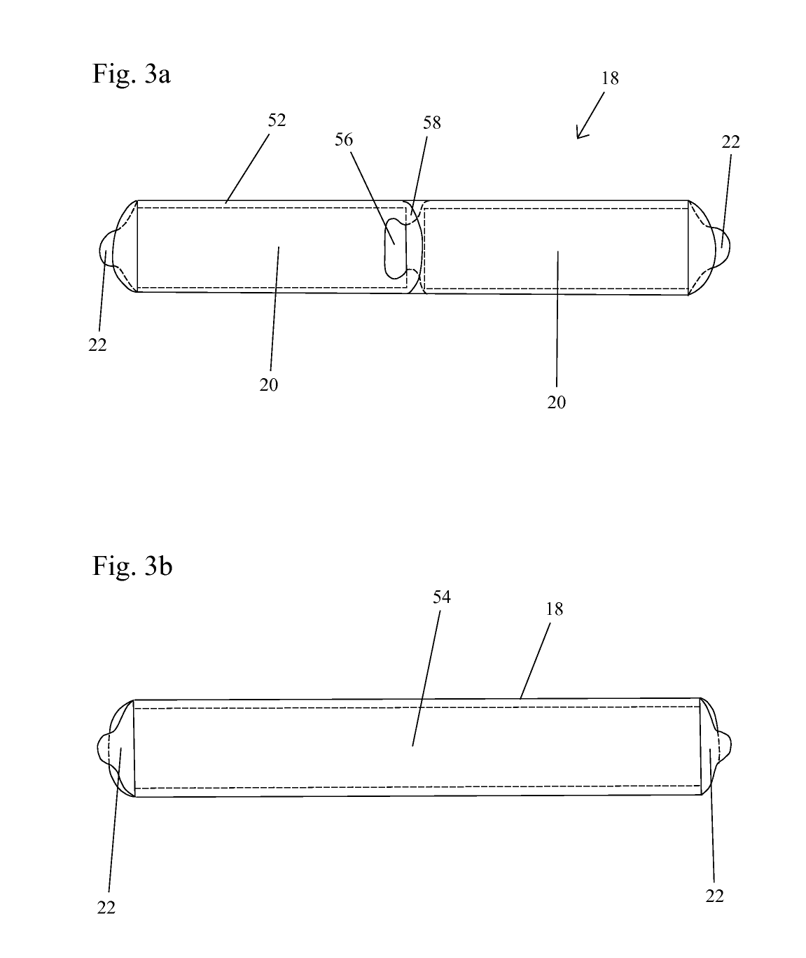

FIG. 3a and FIG. 3b show a front view and a back view, respectively, of the upper lip bar 18. The upper lip bar body 52 extends from the base ring and the adhesive strips 20, 54 are affixed to the upper lip bar body 52. To apply the upper lip bar 18 to the neonate, the upper lip cover strip 22 is removed from the upper lip hydrocolloid gel strip 54, and the upper lip hydrocolloid gel strip 54 is pressed against the skin above the upper lip of the neonate to adhere. The tabbed cheek extension cover strip 56 is then removed from the slotted cheek extension cover strip 58 and the strips 56, 58 are pulled outward to the sides to expose the cheek extension hydrocolloid gel strips 20, which adhere to the cheeks of the neonate to provide additional adhesion between the device and the neonate.

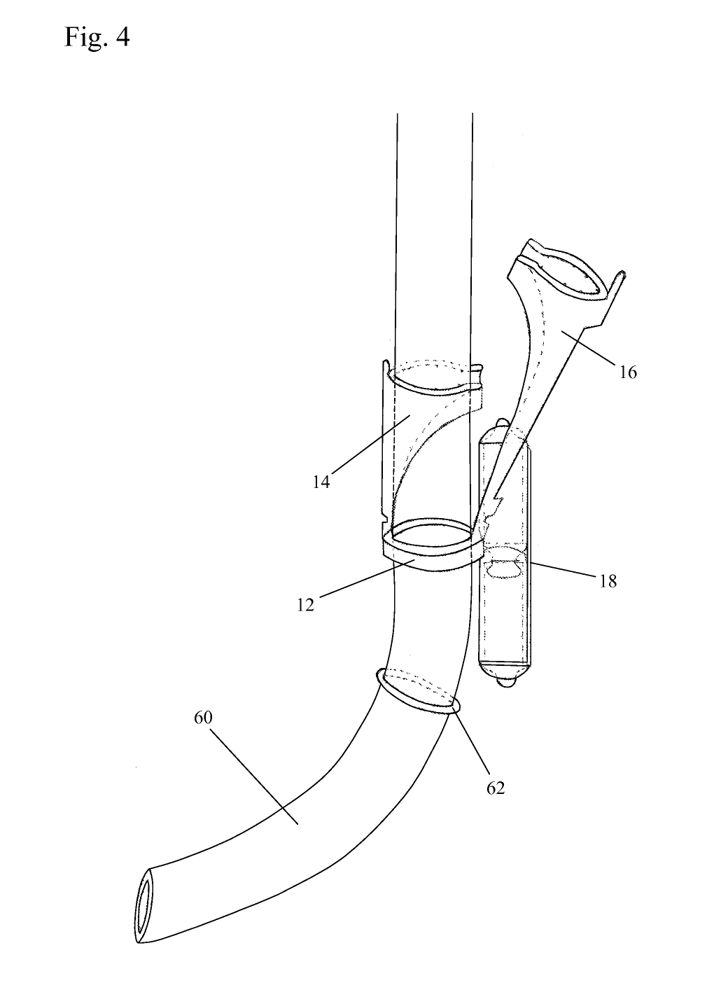

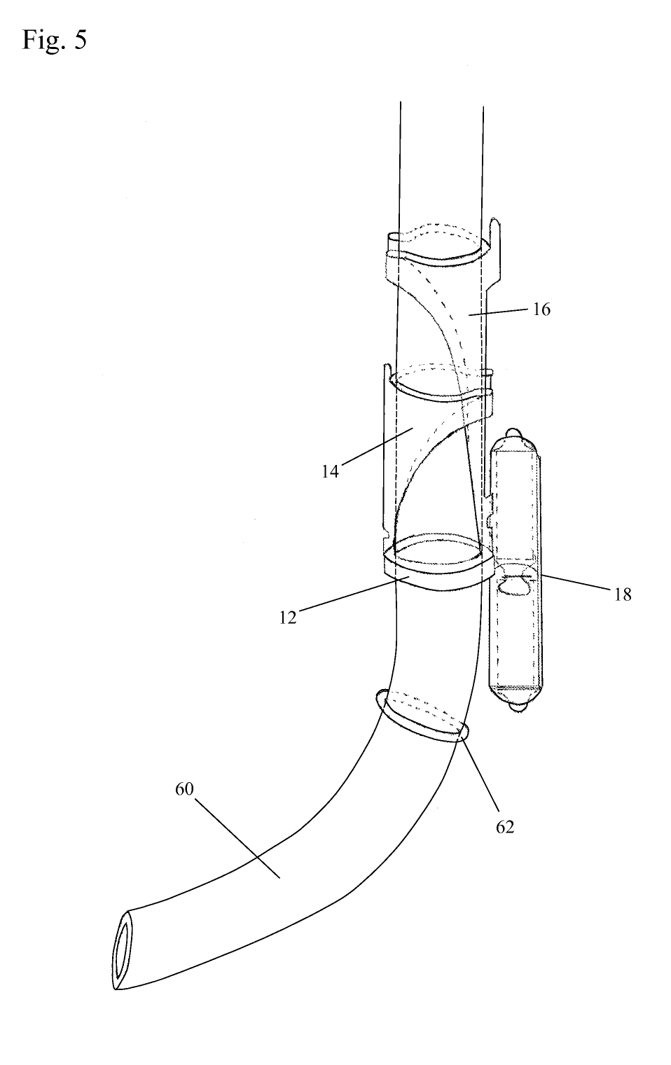

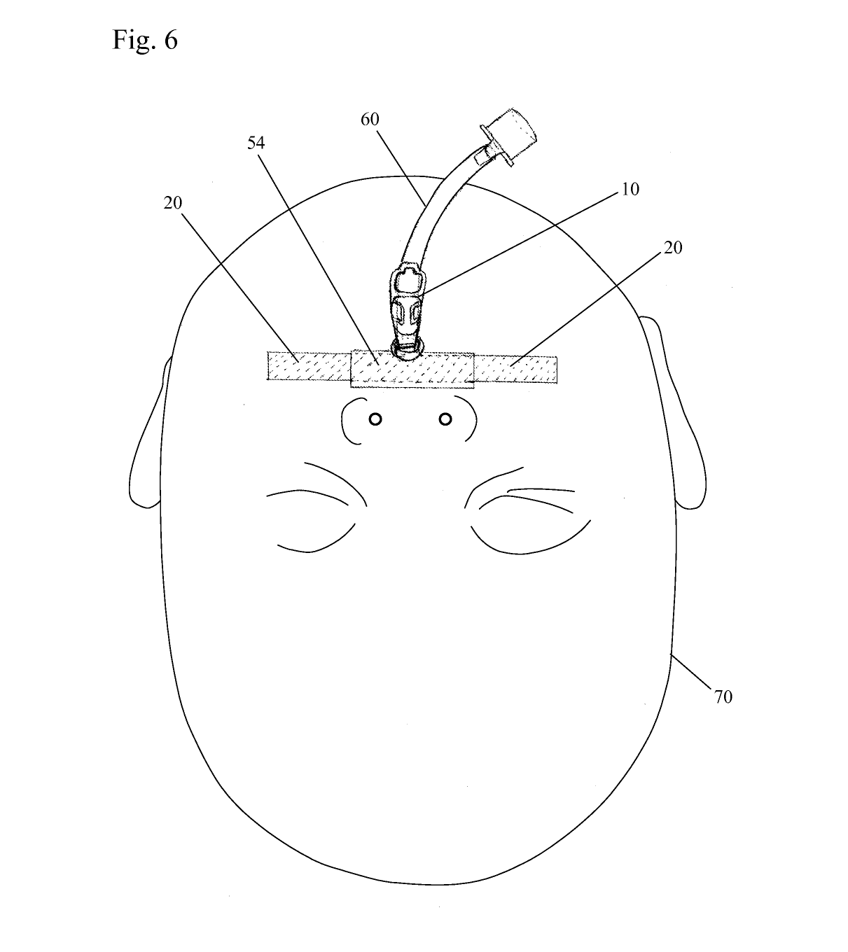

FIG. 4 shows an ET tube 60 with a protection ridge 62 at the 6 centimeter (cm) level. The ET tube 60 has been inserted into the base ring 12 of the secure guard 10 with the short gripper 14 encircling the ET tube 60. FIG. 5 shows the tall gripper 16 also encircling the ET tube 60 such that the secure guard 10 has fully secured the ET tube 60. As shown in FIG. 5, the short gripper 14 and the long gripper 16 are on opposite ends of the base ring 12 facing each other such that the neck of the long gripper 16 goes over and contacts the end portions of the flexible gripper tabs 30, 32 of the short gripper 14 to provide additional support to the short gripper 14. FIG. 1 shows the opening 17 on the neck of the long gripper 16 into which the ends of the flexible gripper tabs 30, 32 of the short gripper 14 extend. In FIG. 6, the ET tube 60, with the secure guard 10 applied to the ET tube 60, has been inserted into the airway of a neonate 70, with the upper lip hydrocolloid gel strip 54 and the cheek extension hydrocolloid gel strips 20 applied to the skin of the neonate 70.

The following are the preferred steps involved in securing the ET tube with a monolithic secure guard:

1. Determine or guess the weight of the baby and choose the appropriate size ET tube with the secure guard in place.

2. As soon as the neonate is intubated to the desired level, the long and short gripper are ungripped from the endotracheal tube, the plastic paper covering the adhesive surface of the hydrocolloid strip facing the upper lip is removed, the secure guard is adjusted based on the intubation formula shown on the upper lip bar, and the horizontal upper lip bar is stuck to the neonate's skin above the upper lip.

3. The plastic paper covering the adhesive strip of the hydrocolloid strip facing away from the upper lip is removed and the cheek extension adhesive strips are attached to the cheeks.

4. The ET tube is adjusted based on the weight of the baby by detaching the short and tall gripper from the ET tube. The ET tube is adjusted, and the short gripper and tall gripper are reattached after the ET tube adjustment has been made.

The following safety precautions are preferably taken:

1. To prevent the accidental dislodging and aspiration of the secure guard, the horizontal upper lip bar is made wider than the mouth, the secure guard is made with a single plastic piece, and the prominent ridge embedded to the wall of the ET tube at 6 cm level prevents accidental slippage of the secure guard.

2. To avoid masking the ET tube markings, the monolithic secure guard is made of a transparent plastic.

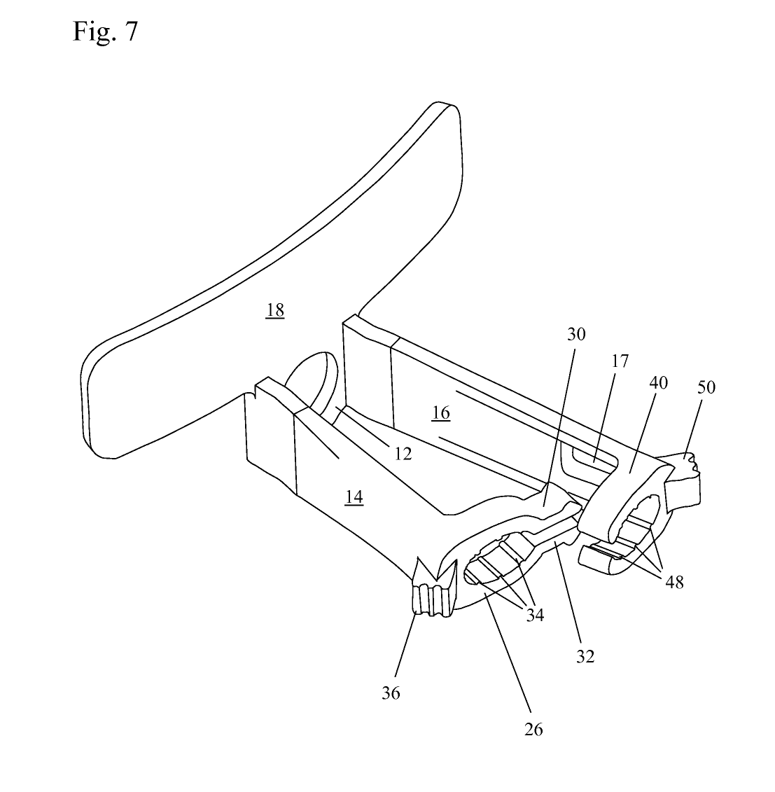

FIG. 7 shows a three-dimensional perspective view of a monolithic secure guard without the hydrocolloid gel strips. The piece shown in FIG. 7 is preferably formed monolithically as a single integral piece from a mold. FIG. 7 shows the horizontal upper lip bar 18 having a shape with ends curving toward the neonate's upper lip. The horizontal upper lip bar 18 extends integrally from the top of the base ring 12. The short gripper 14 and the long gripper 16 extend from the left and right sides, respectively, of the base ring 12 in a direction substantially perpendicularly to the base ring 12 and the horizontal upper lip bar 18 and substantially parallel to each other. The short gripper and long gripper may alternatively extend from the right and left sides, respectively, of the base ring.

FIG. 7 also shows grooves on the tops of the finger tab 36, 50 to provide a gripping surface for the finger when disengaging the gripping portions 26, 40 from the endotracheal tube. The projections 34, 48 extend longitudinally as ridges in the gripping portions 26, 40, respectfully, in FIG. 7. The flexible gripper tabs 30, 32 on the short gripper 14 are designed to extend through the opening 17 on the neck of the long gripper 16, with extensions on the outer surfaces of the tabs 30, 32 to help maintain engagement with the neck of the long gripper 16.

The endotracheal tube with the secure guard may also be adapted for intubation through the nasal route, for example, by using an ET tube without a protection ridge at 6 cm level and locating the base ring at a little higher level than the horizontal upper lip bar.

The endotracheal tube with the secure guard may be tailored to be used in pediatric and adult humans, and small to medium sized pets, while performing endotracheal intubation.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A secure guard for an endotracheal tube comprising:
a base ring having a first opening sized to receive the endotracheal tube;
a first gripper extending from the base ring, the first gripper comprising:
a first flexible neck extending from the base ring; and
a first gripping portion extending from the first flexible neck, the first gripping portion comprising a first gripping projection and a second gripping projection forming a second opening sized to receive the endotracheal tube and separated by a first gap sized to permit lateral insertion of the endotracheal tube into the second opening;
a second gripper extending from the base ring, the second gripper comprising:
a second flexible neck extending from the base ring and having a second length greater than a first length of the first flexible neck; and
a second gripping portion extending from the second flexible neck, the second gripping portion comprising a third gripping projection and a fourth gripping projection forming a third opening sized to receive the endotracheal tube and separated by a second gap sized to permit lateral insertion of the endotracheal tube into the third opening; and
an upper lip bar extending from the base ring, the upper lip bar comprising an upper lip bar body and an upper lip adhesive strip coupled to the upper lip bar body.

2. The secure guard of claim 1, wherein the first gripping portion further comprises a plurality of first projections extending into the second opening and the second gripping portion further comprises a plurality of second projections extending into the third opening.

3. The secure guard of claim 1, wherein the second flexible neck comprises a fourth opening sized to receive the first gripping projection and the second gripping projection when the second opening and the third opening receive the endotracheal tube.

4. The secure guard of claim 1, wherein the base ring, the first gripper, the second gripper, and the upper lip bar body are formed as a single piece.

5. The secure guard of claim 4, wherein the single piece is made of a plastic.

6. The secure guard of claim 1, wherein the first gripper and the second gripper extend from opposite ends of the base ring.

7. The secure guard of claim 1, wherein the adhesive strip comprises a hydrocolloid gel.

8. The secure guard of claim 1, wherein the upper lip bar further comprises a pair of cheek extensions comprising cheek extension adhesive strips.

9. The secure guard of claim 1, wherein the first gripper further comprises a first finger tab extending from the first flexible neck past the first gripper portion and the second gripper further comprises a second finger tab extending from the second flexible neck past the second gripper portion.

10. The secure guard of claim 1, wherein the secure guard is sized to fit a neonate.

11. An endotracheal tube with a secure guard comprising:
the endotracheal tube;
the secure guard comprising:
a base ring having a first opening receiving the endotracheal tube;
a first gripper extending from the base ring, the first gripper comprising:
a first flexible neck extending from the base ring; and
a first gripping portion extending from the first flexible neck, the first gripping portion comprising a first gripping projection and a second gripping projection forming a second opening and separated by a first gap, the second opening receiving the endotracheal tube;
a second gripper extending from the base ring, the second gripper comprising:
a second flexible neck extending from the base ring and having a second length greater than a first length of the first flexible neck; and
a second gripping portion extending from the second flexible neck, the second gripping portion comprising a third gripping projection and a fourth gripping projection forming a third opening and separated by a second gap, the third opening receiving the endotracheal tube; and
an upper lip bar extending from the base ring, the upper lip bar having an adhesive surface.

12. The endotracheal tube with the secure guard of claim 11, wherein the endotracheal tube further comprises a protection ridge.

13. The endotracheal tube with the secure guard of claim 11, wherein the endotracheal tube and the secure guard are sized to fit a neonate.

14. A method of securing an endotracheal tube comprising:
a) inserting a first end of the endotracheal tube through a base ring of a secure guard;
b) placing a first gripping portion of a first gripper extending from the base ring laterally around the endotracheal tube; and
c) placing a second gripping portion of a second gripper extending from the base ring laterally around the endotracheal tube.

15. The method of claim 14, wherein c) further comprises inserting the first gripping portion into an opening in a second flexible neck of the second gripper, the second flexible neck connecting the second gripping portion to the base ring, a first flexible neck connecting the first gripping portion to the base ring, the second flexible neck having a second length greater than a first length of the first flexible neck.

16. The method of claim 14 further comprising inserting a second end of the endotracheal tube opposite the first end of the endotracheal tube into a mouth and a throat of a neonate.

17. The method of claim 16 further comprising adhering an upper lip bar extending from the base ring to a skin surface between an upper lip and a nose of the neonate.

18. The method of claim 16 further comprising:
d) detaching the second gripping portion from the endotracheal tube;
e) detaching the first gripping portion from the endotracheal tube;
f) adjusting the endotracheal tube with respect to the base ring;
g) placing the first gripping portion around the endotracheal tube; and
h) placing the second gripping portion around the endotracheal tube.

19. The method of claim 18, wherein d) through h) occur without removing the upper lip bar from the skin surface.
